(19) United States
(12) Patent Application Publication
(10) Pub. No.: US 2014/0134246 A1
(43) Pub. Date: May 15, 2014

(54) RAPID DISSOLVE TABLET COMPOSITIONS FOR VAGINAL ADMINISTRATION

(71) Applicant: Aptalis Pharmatech, Inc., Vandalia, OH (US)

(72) Inventors: Gopi VENKATESH, Vandalia, OH (US); Vijaya SWAMINATHAN, Vandalia, OH (US); Jin-Wang LAI, Springboro, OH (US)

(73) Assignee: Aptalis Pharmatech, Inc., Vandalia, OH (US)

(21) Appl. No.: 14/069,712

(22) Filed: Nov. 1, 2013

Related U.S. Application Data
(63) Continuation of application No. PCT/US12/36055, filed on May 2, 2012.

(51) Int. Cl. A61K 9/20; A61K 9/00; A61K 31/4164; A61K 31/138; A61K 31/675; A61K 31/513

(52) U.S. Cl. CPC A61K 9/2054; A61K 31/675; A61K 9/2027; A61K 31/513; A61K 31/4164; A61K 31/138; A61K 9/0034; A61K 9/205; A61K 9/2013
USPC 424/465; 514/81; 514/274; 514/398; 514/396; 514/652

(57) ABSTRACT

Disclosed herein are pharmaceutically acceptable rapid dissolve vaginal tablet compositions comprising one or more active pharmaceutical ingredients suitable for therapy via topical action or systemic absorption, and methods of making and using such compositions.

### CROSS REFERENCE TO RELATED APPLICATIONS

The present application claims priority to U.S. Application Ser. No. 61/481,582 filed May 2, 2011, which is incorporated by reference in its entirety for all purposes.

### BACKGROUND OF THE INVENTION

Vaginal drug delivery is a potential route for therapy via topical action or systemic absorption as well as uterine targeting of active pharmaceutical ingredients. It offers advantages such as:
large surface area
avoiding hepatic first-pass effect, which may result in significant enhancement of bioavailability or reduction in dose strength or side-effect profile
dense network of blood vessels
high permeability even for large molecular weight drugs such as peptides and proteins
low systemic drug exposure (namely in the case of products used for local conditions)
low enzymatic activity and the possibility of preferential transfer of absorbed drugs to the uterus (referred to as the “first-uterine-pass effect”)
ease of removal in case of necessity

Despite these advantages, the intravaginal route of administration for drug delivery has been exploited only to a limited extent. Vaginal drug delivery systems such as conventional tablets are traditionally used to deliver contraceptives and microbicides to treat vaginal infection. Vaginal tablets, rings, creams, and viscous gels containing a wide range of drugs (e.g., steroids, prostaglandins, antimicrobials, proteins, and peptides) have been developed, and in vitro, ex vivo, and in vivo performance evaluations of these vaginal drug delivery systems have been studied. Studies of intravaginal drug delivery systems such as these comprising one or more nontoxic, nonirritant, or bioadhesive materials that are easy to incorporate into vaginal dosage forms and which, in theory, could extend the bioactive residence time within the vagina and reduce the dosing frequency as well as the dose administered include: Lehr, 2000. J. Control. Rel. 65, 19-29; Mandal et al. 2000, J. Pharm. Biopharm. 50, 337-343; Garg et al., 2001. Pharma. Tech. 14-23; Ceschel et al., 2001. Drug Del. Ind. Pharm. 6, 541-547; Bilensoy et al., 2006. AAPS PharmSciTech. 7, 19-29; Valenta, 2005. Advanced Drug Del. Rev. 57, 1692-1712; Bonferoni et al., 2006, AAPS Pharm. SciTech. 7, E1-E8; Neves et al., 2008. Eur. J. Pharm. Biopharm. 69, 622-632; Ndesendo et al., 2009. Int. J. Pharm. 370, 151-159; Poelvoorde et al., 2009, J. Pharm. Biopharm. 71, 280-284-343; Perioli et al., 2009. Int. J. Pharm. 377, 120-127; Yellanki et al., 2010. Int. J. PharmTech Res. 2; 1746-1750; and Wang et al., 2002. J. Contr. Rel. 82, 39-50.

A vast majority of bioadhesive gels, creams, and tablets that are used as vaginal delivery systems break down rapidly following application into the vaginal cavity and have minimal bioadherence to the vaginal mucosa. These complications are likely due to miscibility with water or due to a lack of physical stability at body temperature, such that only a limited therapeutic effectiveness is exhibited. Emulsion based vaginal drug delivery systems have been developed, such as ones containing (i) one or more globule stabilizing polymers (e.g., HPMC, polyvinyl alcohol, or a PEGylated lipid) and (ii) a therapeutically active drug; drugs approved for or used for the treatment, prophylaxis, cure, or mitigation of diseases of the vagina, urinary tract, cervix, or other female reproductive organ; inducement of contraception; or systemic drug therapy.

US 20030180366 (U.S. Pat. No. 6,899,890) discloses a novel, microemulsion based, essentially pH neutral, vaginal drug delivery system suitable for modified delivery of a therapeutically active material in the vaginal cavity. US 20050276836 discloses a method of coating a vaginal device for delivering therapeutic or health-promoting agents with a mucoadhesive composition. WO 2008133928 discloses a method of treating a patient having an epithelial lesion, such as of the vagina, or disorder of impaired mucin function as well as methods of treating pain associated with epithelial lesions and disorders of impaired mucin function using a pharmaceutical composition containing mucin glycoproteins in combination with therapeutic agents, e.g., trefoil polypeptides. WO 2010061284 discloses a controlled release, intravaginal, pharmaceutical dosage form consisting of at least one pharmaceutically active ingredient which is admixed with a combination of biocompatible and biodegradable polymers and shaped for insertion into the vagina of a patient. The vaginal administration of a controlled-release opioid such as oxycodone has been shown to be a safe, effective, and simple means of managing cancer pain in patients who cannot tolerate the adverse events caused by oral administration (X. Zhang, X-J. Ruan, C. Liu, and Z-H. Yu, Effect of vaginal administration of controlled-release oxycodone on cancer pain. Chin. J. Cancer J. Cancer, 2009, 28(7) 1-4; F. Acartürk, Mucoadhesive Vaginal Drug Delivery Systems. Recent Patents on Drug Delivery & Formulation 2009, 3, 193-205) (“Acartürk 2009”). Acartürk 2009 recently summarized the development and in vitro/in vivo evaluations of improved formulations for transmucosal vaginal delivery.

Disintegration distribution/spreading, as well as retention time of bioadhesive vaginal dosage forms (e.g., conventional tablet, multiparticulate, viscous gel formulations), have been evaluated extensively. Conventional or even bioadhesive vaginal tablets are easy to administer in privacy by the user; however these dosage forms may be too slow to disintegrate and spread and are cleared from the vagina too rapidly to provide any meaningful improvement in therapy. The level of patient compliance is poor and is generally believed to be influenced by restrictive dosing regimens, a need to consume multiple combination oral drug products, patients' suspicions as to the effectiveness of vaginal therapy, leakage, or discomfort associated with administration. Furthermore, gel dosage forms require the use of a vaginal applicator, thereby resulting in increased packaging materials and manufacturing costs.

One area where vaginal administration has been explored is in the context of the treatment of acquired immune deficiency syndrome (AIDS), as the impact of human immunodeficiency virus (HIV) continues to significantly affect large numbers of worldwide populations. HIV is a retrovirus that primarily infects and directly and indirectly destroys vital components of the human immune system such as CD4⁺ cells (a subset of cells that are required for the proper functioning of the human immune system), macrophages, and dendritic cells. AIDS is a collection of symptoms and infections resulting from specific damage to the immune system caused by HIV in humans. AIDS acquired via sexually transmitted infections (STIs) affect more than 13 million men and women in the USA every year. Although treatments for AIDS and HIV exist to decelerate the virus' progression, there is currently no known cure. Despite a recent report from the Joint United Nations Program on HIV/AIDS (UNAIDS/WHO 2008—Report on the Global AIDS Epidemic. Geneva: UNAIDS, p. 362) claiming the HIV epidemic has stabilized, this past year saw 2.7 million new infections of HIV-1 infection and 2 million more perished.

In patients infected by HIV, the efficacy of highly active antiretroviral (ARV) therapy through the blockade of different steps of the retrovirus life cycle is now well understood. As HIV is a retrovirus that replicates within cells of the immune system, intracellular drug concentrations are important to determine ARV drug efficacy and toxicity. Some ARV agents used for oral administration are prodrugs that require intracellular anabolic phosphorylation to be converted to their active form of triphosphorylated metabolites. The active metabolites, which have longer plasma half-lives than their parent compounds, have been used in the vaginal route of administration.

Some studies in the area of vaginal administration and HIV therapy include the following. In vitro and ex vivo testing of tenofovir (TFV) as a vaginal gel formulation containing 1% TFV and 2% hydroxyethylcellulose (HEC) as a bioadhesive polymer was shown by in vitro and ex vivo testing to be as effective as an HIV-1 microbicide (Mayer et al., 2006. AIDS. 20, 543-551; Rohan et al., PLoS One February 2010. 5, 1-12). New generations of vaginal gels comprising 1% TFV in combination with 5% emitricitabine (FTC) provided protection from Simian HIV exposures in Macaques (Parikh et al., 2009. J. Virology. 83, 10358-10364). The results of studies using (i) dual segment polyurethane vaginal rings sustaining the release of two oral antiretroviral agents, tenofovir and dapivirine, or (ii) vaginal gels containing a thiocarboxanilide UC781, were investigated for antiretroviral activities (Friend, 2000. Pharm. Develop. Technol. 1-20; Mahalingam et al., 2010. Pharm. Res. 27, 2478-2491).

Despite the promising characteristics of the vagina for drug therapy, development and commercialization issues persist, such as:
lack of appropriate in vitro/ex vivo test methods
lack of adequate retention time of the vaginal formulation
lack of adequate spreading characteristics of the vaginal formulation
other limitations include menstrual cycle-associated vaginal changes, genital hygiene issues, local side effects, coitus interference and variable drug permeability
social taboos, unawareness, and gender-specificity are also strong barriers to the use and development of vaginal drug delivery
lack of vaginal formulations having an ease of administration without causing discomfort to improve patient compliance.

The present inventors surprisingly found a way to provide a vaginal dosage form, a ‘rapid dissolve tablet formulation,’ which promises to meet the unmet medical need for a vaginal dosage form that is easy to administer in privacy and which rapidly disintegrates/dissolves upon insertion into the vaginal cavity. This dosage form creates a viscous suspension that is spread rapidly and widely over the vaginal mucosa and is retained for a sufficiently long time to provide therapeutic efficacy via topical action or systemic absorption. Unlike the gel dosage form, use of a vaginal applicator is not required for these tablets, thereby making them an attractive dosage form based on potential reductions in packaging materials (i.e., increased portability) and manufacturing costs. This dosage form may be used in the context of HIV therapy as well as other therapeutic applications.

### SUMMARY OF THE INVENTION

In some embodiments, this invention relates to rapid dissolve tablet compositions comprising one or more active pharmaceutical ingredients for the treatment of disease states via topical action or systemic absorption upon vaginal administration, and methods of making and using such compositions.

In some embodiments, this invention relates to pharmaceutical compositions comprising one or more active pharmaceutical ingredients suitable for vaginal route of administration, and methods of making and using such compositions for therapy via topical action or systemic absorption, as well as uterine targeting. In certain embodiments, the present invention is related to a pharmaceutical composition comprising one or more active pharmaceutical ingredients suitable for vaginal route of administration, one or more polymeric excipients having a dual property of acting as a binder as well as a bioadhesive material, one or more sugar alcohols or saccharides, and one or more disintegrants, which rapidly disintegrates in the vaginal cavity forming a viscous suspension that rapidly and widely spreads to coat the vaginal mucosa with the drug suspension/solution for therapy via topical action or systemic absorption.

The pharmaceutical composition of the present invention may contain at least one drug selected from the group consisting of antifungal agents; antibacterial agents; antimicrobial agents; antiviral agents; anti-infectives; spermicides; hormones; antibiotics; antiviral agents; analgesics; antitrichomonial agents; antiprotozoan agents; antimycoplasm agents; antiretroviral agents; nucleoside analogues; reverse transcriptase inhibitors; protease inhibitors; contraceptive agents; anorexics and appetite suppressants; steroids; anthelmintics; anesthetics; antiarthritics; antiasthma agents; anticonvulsants; antidepressants; antidiabetic agents; antidiarrheals; antihistamines; anti-inflammatory agents; antimigraine preparations; antimotion sickness agents; antinauseants; antineoplastics; antiparkinsonism agents; antipruritics; antipsychotics; antipyretics; antispasmodics; anticholinergics; sympathomimetics; xanthine derivatives; cardiovascular preparations; calcium channel blockers; beta blockers; antiarrhythmics; antihypertensives; diuretics; general, coronary, peripheral and cerebral vasodilators; erectile dysfunction agents; central nervous system stimulants; cough and cold preparations; decongestants; diagnostics; hormones; hypnotics; immunosuppressives; muscle relaxants; parasympatholytics; parasympathomimetics, psychostimulants, sedatives, orally active drugs exhibiting significant first-pass effects; proteins/peptides including growth enhancing hormones and luteinizing-hormone-releasing hormone (LHRH); tranquilizers; antioxidants; vitamins; minerals; and herbal extracts or preparations, or combinations thereof, for vaginal administration.

According to the invention, the bioadhesive property of the polymer excipient (e.g., low-substituted hydroxyethylcellulose, hydroxypropylcellulose, hypromellose, polycarboxylic acids, polyvinylpyrrolidone vinylpyrrolidone-polyvinyl acetate copolymer, ethylene glycol 6000-vinylcaprolactam-vinyl acetate copolymer, polyvinyl alcohol, polyethylene oxide, poly(lactic co-glycolic acid), polyamide, alginic acid salts, carrageenan, chitosan, and cellulosic gum) will enhance bioadherence of the active ingredient (drug) to the mucosa surface, thereby increasing the retention time for improved therapy via topical action or systemic absorption.

In certain embodiments, wherein the pharmaceutical composition could further comprise a surfactant, and/or a lipid that will enhance bioadherence of the active ingredient to the mucosa surface or enhance/sustain systemic absorption, thereby providing improved therapy via topical action or systemic absorption, and reduced side effects; more particularly when the drug has poor water solubility.

In some other embodiments, the present invention is directed to a pharmaceutical composition in the form of a rapid dissolve tablet, which rapidly disintegrates upon insertion into the vagina of a patient, forming a viscous suspension that rapidly and widely spreads and coats the vaginal mucosa with the drug suspension/solution for therapy via topical action or systemic absorption.

Yet in some other embodiments, the present invention is related to a rapid dissolve tablet comprising rapidly dispersing microgranules comprising at least one sugar alcohol and at least one disintegrant. This tablet may rapidly disintegrate upon insertion into the vagina of a patient, forming a viscous drug suspension that rapidly and widely spreads to coat the vaginal mucosa with the drug suspension/solution.

### BRIEF DESCRIPTION OF THE DRAWINGS

FIG. 1 shows a schematic of a ‘Rapid Dissolve Tablet’ as conceived in certain embodiments of the present invention.

FIG. 2 shows mean tenofovir plasma concentration-time profiles following vaginal administration of a single tenofovir rapid dissolve tablet (RDT) in female rabbits.

FIG. 3 shows mean tenofovir plasma concentration-time profiles following a multi-dose (7 once-daily dosing) administration of tenofovir rapid dissolve tablets (RDTs) into the vagina of female rabbits.

FIG. 4 shows mean free and total tenofovir contents of the abdominal and vaginal tissues at 2 and 24 hrs post dosing following vaginal administration of a single tenofovir rapid dissolve tablet (RDT) in female rabbits.

FIG. 5 shows mean free and total tenofovir contents of the abdominal and vaginal tissues at 2 and 24 hrs post dosing following a multi-dose (7 once-daily dosing) administration of tenofovir rapid dissolve tablets (RDTs) into the vagina of female rabbits.

FIG. 6 shows mean predose and postdose tenofovir concentrations in Weck-Cel® at 2 and 24 hrs post dosing following vaginal administration of a single tenofovir rapid dissolve tablet (RDT) in female rabbits.

FIG. 7 shows mean predose and postdose tenofovir concentrations in Weck-Cel® at 2 and 24 hrs post dosing following a multi-dose (7 once-daily dosing) administration of tenofovir rapid dissolve tablets (RDTs) into the vagina of female rabbits.

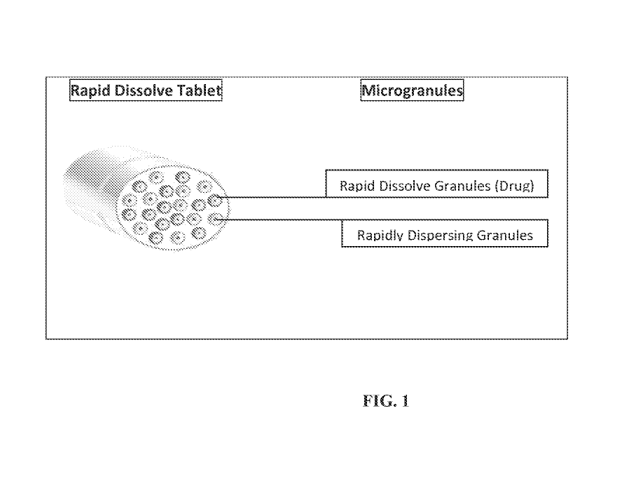

### DETAILED DESCRIPTION OF THE INVENTION

The following description includes information that may be useful in understanding the invention. It is not an admission that any of the information provided herein is prior art or that any publication specifically or implicitly referenced is prior art.

All documents cited herein are incorporated by reference in their entirety for all purposes to the same extent as if each individual document was specifically and individually indicated to be incorporated by reference. As used above, and throughout the description of the invention, the following terms, unless otherwise indicated, shall be understood to have the following meanings:

The term “drug”, “active”, “bioactive material”, “active agent”, or “active pharmaceutical ingredient” as used herein includes a pharmaceutically acceptable and therapeutically effective compound, pharmaceutically acceptable salts, stereoisomers and mixtures of stereoisomers, solvates (including hydrates), polymorphs, or prodrugs thereof. Unless otherwise indicated, when referring to a drug in the descriptions of the various embodiments of the invention, the reference encompasses the base drug, pharmaceutically acceptable salts, stereoisomers and mixtures of stereoisomers, solvates (including hydrates), polymorphs, or prodrugs thereof.

The term “salts” refers to the product formed by the reaction of a suitable inorganic or organic acid with the “free base” form of the drug. Suitable acids include those having sufficient acidity to form a stable salt, for example acids with low toxicity, such as the salts approved for use in humans or animals. Non-limiting examples of acids that may be used to form salts of a vaginally active drug such as metronidazole or tenofovir include inorganic acids, e.g., HF, HCL, HBr, HI, H₂SO₄, H₃PO₄; non-limiting examples of organic acids include organic sulfonic acids, such as C₆₋₁₆ aryl sulfonic acids, C₆₋₁₆ heteroaryl sulfonic acids, or C₁₋₁₆ alkyl sulfonic acids—e.g., phenyl, α-naphthyl, β-naphthyl, (S)-camphor, methyl, ethyl, n-propyl, i-propyl, n-butyl, sec-butyl, i-butyl, t-butyl, pentyl and hexyl sulfonic acids; non-limiting examples of organic acids includes carboxylic acids such as C₁₋₁₆ alkyl, C₆₋₁₆ aryl carboxylic acids, and C₄₋₁₆ heteroaryl carboxylic acids, e.g., acetic, glycolic, lactic, pyruvic, malonic, glutaric, tartaric, citric, fumaric, succinic, malic, maleic, hydroxymaleic, benzoic, hydroxybenzoic, phenylacetic, cinnamic, salicylic and 2-phenoxybenzoic acids; non-limiting examples of organic acids include amino acids, e.g., the naturally-occurring amino acids, lysine, arginine, glutamic acid, glycine, serine, threonine, alanine, isoleucine, leucine, etc. Other suitable salts can be found in, e.g., S. M. Birge et al., J. Pharm. Sci., 1977, 66, pp. 1-19. In most embodiments, “salts” refers to salts that are biologically compatible or pharmaceutically acceptable or non-toxic, particularly for mammalian cells. The salts of drugs useful in the invention may be crystalline or amorphous, or mixtures of different crystalline forms or mixtures of crystalline and amorphous forms.

The term “prodrug” means a form of a compound suitable for administration to a patient without undue toxicity, irritation, allergic response, and the like, and effective for an intended use, including ketal, ester, and zwitterionic forms. A prodrug is transformed in vivo, for example by hydrolysis in blood. A thorough discussion is provided in T. Higuchi and V. Stella, Pro-drugs as Novel Delivery Systems, Vol. 14 of the A. C. S. Symposium Series, and in Edward B. Roche, ed., Bioreversible Carriers in Drug Design, American Pharmaceutical Association and Pergamon Press, 1987.

The terms “rapid dissolve tablet”, “rapid disintegrating tablet”, or “RDT” refer to a tablet that disintegrates rapidly, such as in about 8 min, about 6 min, about 4 min, or about 2 min, in the vaginal cavity of a patient after administration/insertion into the vaginal cavity. The rate of disintegration can vary, but is slower than the rate of disintegration of orally disintegrating tablets, or faster than the rate of disintegration of conventional or bioadhesive vaginal tablets, when tested as described herein (e.g., the USP <701> disintegration time test method).

The term “substantially disintegrates” refers to a level of disintegration amounting to disintegration of at least about 50%, at least about 60%, at least about 70%, at least about 80%, at least about 90%, or about 100% disintegration. The term “disintegration” is distinguished from the term “dissolution” in that “disintegration” refers to the breaking up of or loss of structural cohesion of the constituent particles comprising a tablet, whereas “dissolution” refers to the solubilization of a solid in a liquid (e.g., the solubilization of a drug in solvents or gastric fluids).

The term “water-soluble polymer” refers to a polymer that is soluble (i.e., a significant amount dissolves) in aqueous media, independent of pH.

The term “bioadhesive material” refers to a polymer that improves adherence of the pharmaceutical composition containing a bioadhesive material to mucosa or similar biological surface compared to the adherence of the pharmaceutical composition without the bioadhesive material. Non-limiting examples of bioadhesive materials include bioadhesive polymers such as hydroxypropylcellulose.

The term “patient compliance” refers to non-adherence to dosing regimens by patients who are prescribed to follow a certain dosing regimen of a particular medication in need. Non-compliance or adherence to a dosing regimen is a major medical problem in the world costing billions of dollars and affecting lifestyles of millions of people.

The term “about” used herein in reference to a numerical quantity includes the noted numerical quantity, as well as values near the numerical quantity. For example, “about 60 second” includes 60 seconds, exactly, as well as values close to 60 seconds (e.g., 50 seconds, 55 seconds, 59 seconds, 61 seconds, 65 seconds, 70 seconds, etc.).

The use of the term “or” in the claims is used to mean “and/or” unless explicitly indicated to refer to alternatives only or the alternatives are mutually exclusive. It is specifically contemplated that any listing of items using the term “or” means that any of those listed items may also be specifically excluded from the related embodiment.

Following long-standing patent law, the words “a” and “an,” when used in conjunction with the word “comprising” in the claims or specification, denotes one or more, unless specifically noted.

Unless stated otherwise, the amount of the various pharmaceutically acceptable actives or excipients incorporated into various pharmaceutical compositions in accordance with certain embodiments of the present invention is expressed as the percentage weight of the composition as granulate or RDT. Thus, a 10% of an active in the RDT composition refers to the presence or content of the active in the RDT by 10 weight %.

In some embodiments, the present invention is directed to a pharmaceutical composition comprising a drug selected from the group consisting of antifungal agents; antibacterial agents; antimicrobial agents; antiviral agents; anti-infectives; spermicides; hormones; antibiotics; antiviral agents; analgesics; antitrichomonial agents; antiprotozoan agents; antimycoplasm agents; antiretroviral agents; nucleoside analogues; reverse transcriptase inhibitors; protease inhibitors; contraceptive agents; anorexics and appetite suppressants; steroids; anthelmintics; anesthetics; antiarthritics; antiasthma agents; anticonvulsants; antidepressants; antidiabetic agents; antidiarrheals; antihistamines; anti-inflammatory agents; antimigraine preparations; antimotion sickness agents; antinauseants; antineoplastics; antiparkinsonism agents; antipruritics; antipsychotics; antipyretics; antispasmodics; anticholinergics; sympathomimetics; xanthine derivatives; cardiovascular preparations; calcium channel blockers; beta blockers; antiarrhythmics; antihypertensives; diuretics; vasodilators that are general, coronary, peripheral or cerebral; erectile dysfunction agents; central nervous system stimulants; cough and cold preparations; decongestants; hormones; hypnotics; immunosuppressives; muscle relaxants; parasympatholytics; tranquilizers; antioxidants; vitamins, minerals; and herbal extracts or preparations; parasympathomimetics; psychostimulants; sedatives; orally active drugs exhibiting significant first-pass effects; or combinations thereof, proteins/peptides, reverse transcriptase inhibitors, nucleoside/nucleotide reverse transcriptase inhibitors (NRTIs), non-nucleoside/nucleotide reverse transcriptase inhibitors (NNRTIs), protease inhibitors (PIs), integrase inhibitors, growth enhancing hormones and luteinizing-hormone-releasing hormone (LHRH), and the like, or a pharmaceutically acceptable salt, solvate, or ester thereof, suitable for therapy by vaginal administration via topical action or systemic absorption.

Reverse transcriptase inhibitors RTIs are a class of antiretroviral drug used to treat HIV infection, tumors, and cancer. RTIs inhibit the activity of reverse transcriptase, a viral DNA polymerase enzyme that retroviruses need to reproduce. The mode of action of NRTIs is essentially the same; they are analogues of naturally occurring deoxynucleotides needed to synthesize viral DNA. NNRTIs are compounds that are specifically inhibitory to HIV-1 replication and target HIV-1 reverse transcriptase and have, in addition to the NRTIs and protease inhibitors, gained a definitive place in the treatment of HIV-1 infections. PIs are a class of drugs used to treat or prevent infection by viruses, including HIV and Hepatitis C. Integrase inhibitors (e.g., elvitegravir and MK-2048) are a class of antiretroviral drugs designed to block the action of integrase, a viral enzyme that inserts the viral genome into the DNA of a host cell.

Accordingly, in some embodiments, the present invention is directed to a pharmaceutical composition comprising one or more drugs selected from the group of antiretroviral agents consisting of NRTIs (e.g., apricitabine, entecavir, emtricitabine, tenofovir, abacavir, adefovir, their salts and mixtures thereof), NNRTIs nevirapine, delavirdine, efavirenz, rilpivirine, UC-781, MKE-442, quinoxaline HBY 097, DMP 266, their salts and mixtures thereof), protease inhibitors (e.g., indinavir, amprenavir, darunavir, lotinavir, nelfinavir, ritonavir, sequinavir, atazanavir, tipranavir, their salts and mixtures thereof), and integrase inhibitors (e.g., elvitegravir, MK-2048, their salts and mixtures thereof) for the prevention of initial HIV infection, in addition to treating individuals with HIV/AIDS to contain or eliminate the growth or severity of AIDS. Various commercial vaginal creams, ointments, gels, inserts/rings, and tablets are currently available. For example, Dahl discloses in EP 1773296 the preparation of a pharmaceutical vaginal gel comprising tenofovir. This dosage form may not benefit individuals with AIDS or those who want to avoid the risk of HIV transmission or infections during sexual activity, as the gel/applicator—like other similar gel formulations—may suffer from limitations such as leakage, messiness, and low residence time. However, it is very desirable to provide improved compositions and methods which reduce the risk of HIV transmission or infections during sexual activity.

In certain embodiments, the present invention is directed to a pharmaceutical composition in the form of rapid dissolve microgranules or tablets comprising at least one drug suitable for vaginal administration selected from the group consisting of a bisphosphonate alendronate, clodronate, etidronate, pamidronate, tiludronate, ibandronate, neridronate, risedronate, zoledronic acid, incadronate, minodronate and olpadronate); an anti-migraine drug, such as one selected from the group consisting of ergotamine, dihydroergotamine, ergostine, butalbital, phenobarbital, acetaminophen, diclofenac sodium, ketoprofen, ketorolac, ibuprofen, piroxicam, naproxen, acetylsalicylic acid, flurbiprofen, tolfenamic acid, butorphanol, meperidine, methadone, sumatriptan, naratriptan, razatriptan, zolmitriptan, eletriptan, dexamethasone, hydrocortisone, isometheptene, chlorpromazine, diazepam, droperidol, valproic acid, gabapentin, topiramate and divalproex sodium; an anti-nausea drug, such as one selected from the group consisting of metoclopramide, prochlorperazine, domperidone, ondansetron, tropisetron, dolasetron, nabilone, dronabinol, levonantradol, aprepitant, cyclizine, promethazine, sildenafil, oxytocin, oxytocin, oxybutynin, bromocriptine, rifamycin, azithromycin; steroids used in hormone replacement therapy or for contraception; calcitonin; LHRH and analogues; insulin; and human growth hormones, and combinations thereof. Vaginally administratable dosage forms may be employed, such as those including suppositories that contain poorly soluble/bioavailable drugs and effervescent agents as penetration enhancers, and one or more mucoadhesive polymers (e.g., carbomers, chitosan, hydroxyethylcellulose), surfactants (e.g., glyceryl palmitostearate), or lipids (e.g., glyceryl palmitostearate, phospholipid).

In certain embodiments, the present invention is directed to a pharmaceutical composition in the form of rapid dissolve microgranules comprising at least one drug suitable for vaginal administration, at least one sugar alcohol such as mannitol or a saccharide such as lactose, and at least one polymeric binder such as low-substituted hydroxyethylcellulose. Such compositions may be used, for example, for therapy via topical action or systemic absorption upon insertion of the medicinal composition into the vaginal cavity of a patient/subject in need thereof.

In certain embodiments of the present invention, the pharmaceutical composition in the form of rapid dissolve microgranules further comprises a disintegrant such as crospovidone. This disintegrant may facilitate rapid disintegration of rapid dissolve microgranules to forma viscous drug-containing suspension in the vaginal cavity of a patient/subject in need thereof.

In certain other embodiments of the present invention, the pharmaceutical composition in the form of rapid dissolve microgranules further comprises at least one bioadhesive polymer, such as low-substituted hydroxyethylcellulose. This bioadhesive polymer may be provided at a desired concentration, which may, upon forming a viscous drug-containing suspension in the vaginal cavity of a patient/subject, coat the vaginal mucosa, thereby improving its bioadherence and therapeutic efficacy via longer retention for topical action or systemic absorption.

Examples of sugar alcohols/saccharides include, but are not limited to, mannitol, sorbitol, xylitol, arabitol, erythritol, glycerol, hydrogenated starch hydrolysate, isomalt, lactitol, lactose, maltitol, sucrose, maltose, and combinations thereof.

Examples of suitable binders include, but are not limited to, polyvinylpyrrolidone (PVP), polyethylene oxide, hydroxypropyl methylcellulose or hypromellose (e.g., Methocel E5 or E15, or Pharmacoat™ 603), hydroxypropylcellulose Klucel® LF), low-substituted hydroxyethylcellulose, and polysaccharides. The binder can be present in an amount ranging from, e.g., about 0.5-3 weight % based on the rapid dissolve microgranules.

Examples of disintegrants include, but are not limited to crospovidone, sodium starch glycolate, starch, crosslinked sodium carboxymethylcellulose, low-substituted hydroxypropylcellulose, gums (e.g., gellan gum), and combinations thereof. The disintegrant can be present in the pharmaceutical composition in the form of rapid dissolve microgranules, for example, from about 1% to about 10%, from about 3% to about 7%, to about 5%, inclusive of all ranges and subranges there between.

Non-limiting examples of suitable bioadhesive polymers include, but are not limited to, hydroxypropylcellulose, hypromellose, low-substituted hydroxyethylcellulose, hydroxyethylethylcellulose, polycarboxylic acids, polyvinylpyrrolidone, vinylpyrrolidone-polyvinyl acetate copolymer (e.g., Kollidon® VA 64 from BASF), polyvinyl alcohol, polyethylene oxide, carbomers (CARBOPOL® 974P, 941, 940, 934, G70), poly(lactic co-glycolic acid), polyamide, carrageenan, chitosan, and various cellulosic gums (e.g., xanthan gum). The bioadhesive polymer can be present in the pharmaceutical composition in the form of rapid dissolve microgranules, from, for example, about 3% to about 10%, from about 4% to about 8%, to about 5%, inclusive of all ranges and subranges there between.

Non-limiting examples of suitable surfactants that may be employed include tocopherol, surfactants (e.g., CAPTEX 200, Tween 20, Tween 80, Vitamin E TPGS, Capryol 90, CREMOPHOR EL, CARBITOL, PEG 400, lecithin, Brij 92, LABRASOL, triacetin, sodium lauryl sulfate, ethylene glycol monostearate, polysorbates and poloxamers®, GELUCIRE®, LABRAFIL®, LABRASOL®, IMWITOR®, sodium lauryl salicylate, sodium dodecyl sulfate), and mixtures thereof.

Non-limiting examples of suitable lipids that may be employed include lecithins; hydrogenated lecithins; lysolecithin, hydrogenated lysolecithins; lysophospholipids and derivatives thereof; phospholipids and derivatives thereof; salts of alkylsulfates; salts of fatty acids; sodium docusate; stearyl alcohol, glyceryl palmitostearate (PRECIROL® ATO 5); mixtures of mono-, di-, and tri-esters of glycerol (GELUCIRE®), mono- and di-esters of PEG, free PEG, and mixtures thereof.

In certain embodiments of the present invention, a pharmaceutical composition in the form of a rapid dissolve tablet further comprises rapidly dispersing micro comprising a saccharide or a sugar alcohol in combination with a disintegrant, produced in accordance with the specifications co-pending U.S. patent application Ser. No. 10/827,106 (published as US 2005/0232988). Yet in certain other embodiments of the present invention, the rapid dissolve tablet composition can further comprise rapidly dispersing microgranules comprising a sugar alcohol such as mannitol, a super disintegrant such as low-substituted hydroxypropylcellulose, and an additive with multi-functionality of a binder and disintegrant such as starch, modified starch and hydroxypropylcellulose.

Suitable disintegrants include, but are not limited to, crospovidone, sodium starch glycolate, starch, crosslinked sodium carboxymethylcellulose, low-substituted hydroxypropylcellulose, gums (e.g., gellan gum), and combinations thereof. Exemplary saccharides or sugar alcohols may be selected from the group consisting of arabitol, erythritol, glycerol, hydrogenated starch hydrolysate, isomalt, lactitol, lactose, maltitol, mannitol, sorbitol, xylitol, sucrose, maltose, and combinations thereof. The saccharide or sugar alcohol may also be supplemented or replaced with artificial sweeteners such as sucralose. The ratio of the disintegrant to the saccharide or sugar alcohol in the rapidly dispersing microgranules typically ranges from about 1:99 to about 10:90, or from about 5:95 to about 10:90 on a weight basis and inclusive of all ranges and subranges there between. In some embodiments, the disintegrant or the saccharide or sugar alcohol, or both, are present in the form of particles having an average particle size of about 30 μm or less in accordance with the specifications in co-pending U.S. patent application Ser. No. 10/827,106 (published as US 2005/0232988) and where the composition has a multifunctional additive the saccharide or sugar alcohol, are present in the form of particles having an average particle size of about 60 μm or less. The multifunctional additive may be present in the rapidly dispersing microgranule composition at 1-2.5% by weight, for example. The ratio of the drug-containing granules to the rapidly disintegrating granules can range from about 5:1 to about 1:5, from about 3:1 to about 1:3, or from about 2:1 to about 1:2, or about 1:1, inclusive of all ranges and subranges there between.

The in vitro dissolution testing of pharmaceutical compositions of the present invention is performed using United States Pharmacopeia I (paddles at 100 rpm) or II (paddles at 50 rpm) and an appropriate dissolution media (900 mL) (HPLC method) depending on the drug(s).

Disintegration of the RDTs of the invention is tested according to the USP <701> Disintegration Test. Alternately, disintegration time of pharmaceutical compositions prepared as RDT tablets may be determined using a vaginal fluid stimulant prepared in accordance with the disclosure by Owen and Katz (Owen and Katz, 1999, Contraception. 59, 91-95). In view of the small volume of fluid available in the vaginal cavity, dissolution testing may be performed by dropping RDTs in test tubes containing a small amount of buffer (e.g., 3.5 mL) of ammonium acetate buffer at a pH of about 6, and at appropriate time points centrifuging at 4000 rpm for 2 min, and testing samples filtered through 0.45 μm PTFF filters by HPLC.

In a particular embodiment, an RDT of the invention comprises a therapeutically effective amount of tenofovir, or pharmaceutically acceptable salts thereof, alone or in combination with emitricitabine at a ratio of from about 2:1 to about 1:10, from about 1:1 to about 1:8, or from about 1:2 to about 1:6, or about 1:5. After insertion into the vagina, the RDT substantially disintegrates in the vaginal cavity of a patient, forming a viscous, easy-to-spread suspension that spreads/coats the vaginal mucosa to provide efficacy via topical action or systemic absorption. In addition to the rapidly dispersing microgranules, an RDT of the invention optionally includes a pharmaceutically acceptable bioadhesive polymer, such as one selected from the group consisting of low-substituted hydroxyethylcellulose, hydroxypropylcellulose, hypromellose, polycarboxylic acids, polyvinylpyrrolidone, vinylpyrrolidone-polyvinyl acetate copolymer (e.g., Kollidon® VA 64 from BASF), SOLUPLUS®, poly(ethylene glycol 6000-vinylcaprolactam-vinyl acetate) (13:57:30) copolymer from BASF), polyvinyl alcohol, polyethylene oxide, polylactic co-glycolic acid), polyamide, alginic acid salts, carrageenan, chitosan, and various cellulosic gums (e.g., xanthan gum).

In some embodiments, an RBI weighs not less than about 50 mg; for example, 100 mg or more; 200 mg or more; 300 mg or more; or 500 mg or more. In some other embodiments, the RDT weighs not more than about 2000 mg; for example, 1600 mg or less; 1400 mg or less; 1200 mg or less; 1000 mg or less; 800 mg or less; or 500 mg or less. In another embodiment, the RDT weighs not more than about 800 mg. In another embodiment, the RDT weighs not more than about 600 mg. In another embodiment, the RDT weights not more than 500 mg. The dosage forms of the invention can, for example, comprise two or more populations of antibiotic drug-containing particles, such as including at least one population of metronidazole particles as described herein. A dosage form can, for example, comprise a population of tenofovir, nucleoside reverse transcriptase inhibitor, rapid dissolve particles as described herein, and in addition, a population of emitricitabine particles, for the prevention of AIDS.

In one embodiment, a treatment as described herein targets two specific viral enzymes: reverse transcriptase (e.g., using NRTIs or NNRTIs) and protease (e.g., using protease inhibitors).

A pharmaceutical composition of the present invention in the form of a rapid dissolve tablet for vaginal administration may comprise a therapeutically effective amount of propranolol, a non-selective beta blocker undergoing extensive first-pass (hepatic) metabolism upon oral administration, or pharmaceutically acceptable salt or a mixture thereof.

A pharmaceutical composition of the present invention in the form of a rapid dissolve tablet for vaginal administration may comprise a therapeutically effective amount of metronidazole, and optionally an antibiotic selected from the group consisting of clarithromycin, sulfonamides, erythromycin, azithromycin, doxycycline, quinolones, cefoxitin, ceftriaxone cifrofloxacin, doxyclycline, vancomycin, clindamycin, rifaximin, and metronidazole.

A pharmaceutical composition of the present invention in the form of a rapid dissolve tablet for vaginal administration may comprise a therapeutically effective amount of clotrimazole, and optionally an antifungal agent selected from the group consisting of nystatin, ketoconazole, itraconazole, and clotrimazole.

In certain embodiments, the present invention is directed to a method of preparing first a rapid dissolve microgranule composition comprising at least one sugar alcohol, saccharide, or mixture thereof, a polymeric binder, optionally a super disintegrant or bioadhesive polymer, and a therapeutically effective amount of at least one drug selected from the group consisting of antifungal agents, antibacterial agents, antimicrobial agents, antiviral agents, spermicides, hormone agents, antitrichomonial agents, antiprotozoan agents, antimycoplasm agents, antiretroviral agents, nucleoside analogues, reverse transcriptase inhibitors, protease inhibitors, contraceptive agents, proteins, peptides, steroids, growth enhancing agents, and the like, or a pharmaceutically acceptable salt, solvate, or ester thereof, suitable for administration by the vaginal route, next blending drug-containing rapid dissolve microgranules with rapidly dispersing microgranules prepared in accordance with the specifications in co-pending U.S. patent application Ser. No. 10/827,106 (published as US 2005/0232988, and compressing into rapid dissolve tablets (RDTs) containing a therapeutically effective dose. The RDT rapidly disintegrates in the vaginal cavity of a patient/subject forming a viscous drug-containing suspension which is expected to spread rapidly and widely coating the vaginal mucosa for the treatment of the disease via topical action or systemic absorption.

In certain other embodiments, the present invention is directed to a method of preparing first a rapid dissolve microgranule composition comprising at least one sugar alcohol, saccharide, or mixture thereof, a polymeric binder, optionally a super disintegrant or bioadhesive polymer, and a therapeutically effective amount of at least one drug selected from the group of antiretroviral agents consisting of NRTIs (e.g., apricitabine, entecavir, emtricitabine, tenofovir, abacavir, adefovir, their salts and mixtures thereof), NNRTIs (e.g., nevirapine, delavirdine, efavirenz, UC-781, MKC-442, quinoxaline HBY 097, DMP 266, their salts and mixtures thereof), protease inhibitors (e.g., indinavir, amprenavir, darunavir, lotinavir, nelfinavir, ritonavir, sequinavir, atazanavir, tipranavir, their salts and mixtures thereof), and integrase inhibitors (e.g., elvitegravir, MK-2048, their salts and mixtures thereof), next blending drug-containing rapid dissolve microgranules with rapidly dispersing microgranules, and compressing into RDT tablets containing a therapeutically effective dose for the prevention of initial infection of in addition to treating individuals with, HIV/AIDS, such as to contain or eliminate the growth or severity of AIDS. Various commercial vaginal creams, ointments, gels, inserts/rings, and tablets are currently available. For example, Dahl discloses in EP 1773296 the preparation of a pharmaceutical vaginal gel comprising tenofovir. This dosage form may not benefit individuals with AIDS or those who want to avoid the risk of HIV transmission or infections during sexual activity, as the gel/applicator—like other similar gel formulations—may suffer from limitations such as leakage, messiness, and low residence time. However, it is very desirable to provide improved compositions and methods which reduce the risk of HIV transmission and/or infections during sexual activity.

In certain other embodiments, the present invention is related to a method of preparing a rapid dissolve tablet composition comprising at least one sugar alcohol such as mannitol, a polymeric binder such as low-substituted hydroxypropylcellulose, optionally a disintegrant such as crospovidone, and one NRTI alone, such as tenofovir or emtricitabine, or one or more NRTIs, such as tenofovir in combination with emtricitabine or dapivirene, and then form rapid dissolve tablets by compressing a formulation comprising said rapid dissolve microgranules, rapidly dispersing microgranules, microcrystalline cellulose, a super disintegrant such as crospovidone, and a lubricant such as sodium stearyl fumarate on a rotary tablet press.

In another embodiment, a method of preparing a rapid dissolve microgranules comprises granulating a composition as described herein, further comprising a bioadhesive polymer (e.g., low-substituted hydroxyethylcellulose) for incorporation into an RDT, which improves bioadherence of the viscous drug suspension to the surface of the vaginal mucosa upon insertion of the RDT into the vaginal cavity.

The granulation method is not limited; a fluid bed or high shear granulation method using a solution of a polymeric binder dissolved in purified water, ethanol, isopropanol, acetone, or a mixture thereof, is an embodiment of the present invention. In accordance with the present invention, for example, granulation may be performed by spraying a solution comprising a polymeric binder and a drug dissolved or homogeneously suspended therein onto the powder mixture comprising at least one sugar alcohol and optionally a disintegrant or a bioadhesive polymer such as low-substituted hydroxyethylcellulose in a top spray fluid bed granulator such as Glatt GPCG 3, GPCG 5, GPCG 120, or Fluid Air FA0300, and drying the granulation in the same fluid-bed dryer. The granulation may also be performed using a high shear granulator, such as GMX 25 (batch size: 4-7 kg), GMX 65, or GMX 600 (batch size: 140-160 kg) from Vector and drying in the Glatt. The dried granulation thus produced may be sieved by passing through appropriate sieves to collect rapid dissolve drug-containing microgranules with a desired particle size distribution by discarding fines and optionally milling/resieving oversized granules.

The drug-containing microparticles granulated with one or more bioadhesive polymers to improve bioadherence characteristics to the vaginal mucosa may have a median particle size in the range of about 100-400 μm. In some embodiments, not less than 90% of the microparticles are smaller than 600 μm for their incorporation into a rapid dissolve tablet.

In yet another embodiment, the invention may be directed to a method of preparing a rapid dissolve tablet by blending rapid dissolve drug-containing microgranules and rapidly dispersing microgranules prepared as described herein and compressing on a rotary tablet press into rapid dissolve tablets for administration into vaginal cavity of a patient in need of such a medication for therapeutic efficacy via topical action or systemic absorption.

A rapid dissolve tablet of the present invention can be produced by an internal lubrication method, for example, wherein the compression mix is further blended with a lubricant prior to compression. Alternately, a rapid dissolve tablet can be produced by an external lubrication method wherein a lubricant is not included in the tablet formulation, but is externally applied onto the material contacting surfaces of punches and dies of a rotary tablet press. Lubricants such as magnesium stearate, calcium stearate, zinc stearate, stearic aid, sodium stearyl fumarate, glyceryl behenate, and the like may be used for lubricating the granules, or may be externally applied onto material contacting die and punch surfaces of a rotary tablet press used to compress tablets.

Another embodiment according to the invention is directed to a method of treating a patient or subject comprising administering a composition of the invention as a rapid dissolve tablet containing a therapeutically effective amount of one or more drugs selected from the group consisting of antifungal agents, antibacterial agents, antimicrobial agents, antiviral agents, spermicides, hormones, antitrichomonial agents, antiprotozoan agents, antimycoplasm agents, antiretroviral agents, nucleoside analogues, reverse transcriptase inhibitors, protease inhibitors, contraceptive agents, steroids, orally active drugs exhibiting significant first-pass effects, proteins/peptides including growth enhancing hormones and luteinizing-hormone-releasing hormone (LHRH), by insertion into the vaginal cavity of a patient in need thereof.

### EXAMPLES

The invention is described in greater detail in the sections below. Many of the examples provided below to illustrate the invention involve tenofovir alone, or in combination with emtricitabine. It should be understood that, the examples and embodiments described herein are for illustrative purposes only and that various modifications or changes in light thereof will be suggested to persons skilled in the art and are to be included within the spirit and purview of this application.

### Example 1

A: RD Microgranules Comprising Tenofivir, Crospovidone, and Klucel:

Sodium bicarbonate (54 g) is slowly added to purified water (2800 g) in a stainless steel container while continuously stirring to dissolve. The pH of the bicarbonate solution is adjusted to about 6.0 if needed by adding hydrochloric acid. Hydroxypropylcellulose (Klucel LF; 125 g) is slowly added while stirring to dissolve; then tenofovir (180 g) is added to dissolve. Preheated Glatt GPCG 3 equipped with top spray insert, granulation air distribution bottom plate, 200 mesh product retention screen, and 1.0 mm spray nozzle is charged with mannitol with an average particle size of less than 30 μm (781 g) and crospovidone (60 g), both deagglomerated by passing through Comil. The rapid dissolve microgranule composition is granulated while fluidizing the charge continuously and maintaining the process parameters at the following conditions: product temperature—34±1° C.; fluidization air flow—10 CFM; spray rate—10-16 mL/min. Upon completion of spraying, the RD microgranules are dried for a loss on drying to about 1% by weight.

B: RD Microgranules Comprising Tenofivir, Crospovidone, and L-HEC:

Sodium bicarbonate (54 g) is slowly added to purified water (2500 g) in a stainless steel container while continuously stirring to dissolve. The pH of the bicarbonate solution is adjusted to about 6.0 if needed by adding hydrochloric acid. Low-substituted hydroxyethylcellulose (L-HEC; 20 g) is slowly added while stirring to dissolve; then tenofovir (180 g) is added to dissolve. Preheated Glatt GPCG 3 is charged with deagglomerated mannitol (781 g) and crospovidone (60 g) and fluidized. The RD microgranules are prepared by spraying the solution as disclosed in step Ex. 1A above.

C: Microgranules Comprising Tenofivir, Crospovidone, and HPMC:

Sodium bicarbonate (39.1 g) is slowly added to purified water (2500 g) in a stainless steel container while continuously stirring to dissolve. The pH of the bicarbonate solution is adjusted to about 6, if needed by adding hydrochloric acid. Hypromellose (HPMC; 18.1 g) is slowly added while stirring to dissolve; then tenofovir (183.2 g) is added to dissolve. Preheated Glatt GPCG 3 is charged with deagglomerated mannitol (637.4 g) and crospovidone (43.1 g) and fluidized. The RD microgranules are prepared by spraying the solution as disclosed in step Ex.1A above

D. Rapidly Dispersing Microgranules:

Rapidly dispersing microgranules are prepared following the procedure disclosed in co-pending US Patent Application Publication No. U.S. 2003/0215500. Specifically, D-mannitol (152 kg) with an average particle size of approximately 20 μm or less (Pearlitol 25 from Roquette, France) is blended with 8 kg of cross-linked povidone (Crospovidone XL-10 from ISP) in a high shear granulator (GMX 600 from Vector), granulated with purified water (approximately 32 kg), wet-milled using a Comil from Quadro, and finally tray-dried to provide microgranules having an LOD (loss on drying) of less than about 0.8%. The dried granules are sieved, and oversize material is again milled to produce rapidly dispersing microgranules with an average particle size in the range of approximately 175-300 μm.

E: Tenofivir RDTs (Crospovidone):

RDT tablet formulations containing tenofovir (TFV) RI) microgranules of Ex. 1A, Ex. 1B, and Ex. 1C are compressed (see Table 1 for compositions) on a Hata tablet press equipped with partial tooling at the turret speed of 15 RPM. Half of the rapidly dispersing microgranules is charged into a 0.25 cu-ft V blender, followed by crospovidone, microcrystalline cellulose (Avicel PH101), TFV RD granules, and the remaining half of the rapidly dispersing microgranules and blended for 10 min. Sodium stearyl fumarate (Pruv) passed through 35 mesh screen is added to the compression mix and blended 2 min. The Hata tablet press is set up in the manual mode for a fill weight of 150 mg and hardness of 10-50 N. Once the set up is complete, the tablet press is run at a compression force of 3, 4, and 5 kN in the ‘Auto’ mode. Tablets are collected at steady state for each compression force for testing of weight, thickness, hardness, and friability. The average values tested for each tablet batches are presented in Table 1. The disintegration times tested in accordance with USP method <701> for DT are 60-90 sec, 30-60 sec, and 30-60 sec, respectively for tenofovir RDT tablet batches of Formula Ex. 1AD (TFV RDG: Ex. 1A), Formula Ex. 1BD (TFV RDG: Ex. 1B), and Formula Ex. 1CD (TFV RDG: Ex. 1C). RDTs of these formulations compressed at 4 kN are observed to disintegrate in 30-60 sec when tested by the USP method <701> and not less than 80% dissolved at 30 min when tested for dissolution in test tubes.

TABLE 1
Compositions of Tenofovir RDTs

| | Formula Ex. 1AD | | Formula Ex. 1BD | | Formula Ex. 1CD | |
|---|---|---|---|---|---|---|
| | (TFV RDG: Ex. 1A) | | (RDG: Ex. 1B) | | (RDG: Ex. 1C) | |
| Ingredients | mg/tablet | g/batch | mg/tablet | g/batch | mg/tablet | g/batch |
| TFV RD Microgranules | 66.67 | 888.9 | 66.67 | 888.9 | 108.70 | 942.0 |
| Rapidly Dispersing | 58.58 | 781.1 | 58.58 | 781.1 | 16.55 | 143.5 |
| Granules | | | | | | |
| Crospovidone | 7.5 | 100.0 | 7.5 | 100.0 | 7.5 | 65.0 |
| Avicel PH101 | 15.0 | 200.0 | 15.0 | 200.0 | 15.0 | 120.0 |
| Sodium stearyl fumarate | 2.3 | 30.0 | 2.3 | 30.0 | 2.3 | 19.5 |
| Total | 150.0 | 2000 | 150.0 | 2000 | 150.0 | 1300 |

| Parameter | | | | | | | | | |
|---|---|---|---|---|---|---|---|---|---|
| Compression | Formula Ex. 1AD | | | Formula. Ex. 1BD | | | Formula Ex. 1CD | | |
| Force | 3 kN | 4 kN | 5 kN | 3 kN | 4 kN | 5 kN | 3 kN | 4 kN | 5 kN |
| Weight (mg) | 152 | 154 | 149 | 153 | 153 | 151 | 149 | 148 | 154 |
| Thickness, mm | 3.94 | 3.73 | 3.52 | 3.89 | 3.69 | 3.51 | 3.82 | 3.67 | 3.58 |
| Hardness, N | 17 | 35 | 36 | 13 | 24 | 36 | 13 | 20 | 39 |
| Friability | 0.4% | 0.11% | 0.17% | 0.51% | 0.29% | 0.26% | 0.58% | 0.23% | 0.13% |

### Example 2

A: RD Microgranules Comprising Tenofivir:

Sodium bicarbonate (67.5 g) is slowly added to purified water (2010 g) in a stainless steel container while continuously stirring to dissolve. The pH of the bicarbonate solution is measured to be about 6.8. Hypromellose (Methocel; 62.5 g) is slowly added while stirring to dissolve; then tenofovir (190 g) is added to dissolve. Preheated Glatt GPCG 3 is charged with deagglomerated mannitol (890 g) and fluidized. The rapid dissolve microgranules are prepared while spraying the charge and maintaining the process parameters at the following conditions: product temperature—34±1° C.; fluidization air flow—5 to 15 CFM; spray rate—4-16 mL/min. Upon completion of spraying, the RD microgranules are dried for a loss on drying to about 1% by weight.

B: RD Microgranules Comprising Tenofivir:

Sodium bicarbonate (57 g) is slowly added to a mixture of ethanol (540 g) and purified water (1260 g) in a stainless steel container while continuously stirring to dissolve. The pH of the bicarbonate solution is measured to be about 6.2. Next, tenofovir (200 g) is added to dissolve followed by the addition of Low-substituted hydroxyethylcellulose (L-HEC; 20 g) to dissolve while stirring. Preheated Glatt GPCG 3 is charged with deagglomerated mannitol (1023 g) and fluidized. The RD microgranules are prepared by spraying the solution at the following conditions: product temperature—34±1° C.; fluidization air flow—4 CFM; spray rate—8-12 mL/min. Upon completion of spraying, the RD microgranules are dried for a loss on drying to about 1% by weight.

C: RD Microgranules Comprising Tenofivir:

Sodium bicarbonate (54 g) is slowly added to a mixture of ethanol (540 g) and purified water (1260 g) in a stainless steel container while continuously stirring to dissolve. The pH of the bicarbonate solution is measured to be about 6.15. Next, tenofovir (200 g) is added to dissolve followed by the addition of Low-substituted hydroxyethylcellulose (L-HEC; 30 g) to dissolve while stirring. Preheated Glatt GPCG 3 is charged with deagglomerated mannitol (1023 g) and fluidized. The RD microgranules are prepared by spraying the solution as disclosed in step Ex.2B above. Upon completion of spraying, the loss on drying of RD microgranules is about 1.5% by weight.

D. Tenofivir RDTs:

RDT tablet formulations (Formula Ex. 2BD and Formula Ex. 2CD) containing tenofovir (TFV) RD microgranules of Ex. 2B and Ex. 2C are compressed (see Table 2 for compositions) on a Hata tablet press equipped with partial tooling at the compression force of 4 kN and turret speed of 15 RPM. Samples are collected at the start, mid, and end of the run for testing for in-process tablet properties. The results are presented in Table 2. In case of Formula Ex. 2AD, tablets are compressed using a Carver press.

TABLE 2
Compositions of Tenofovir RDTs

| | Formula | | Formula | | Formula | |
|---|---|---|---|---|---|---|
| | Ex. 2AD* | | Ex. 2BD | | Ex. 2CD | |
| | (TFV RDG: | | (TFV RDG: | | (TFV RDG: | |
| | Ex. 2A) | | Ex. 2B) | | Ex. 2C) | |
| | mg/ | | mg/ | | mg/ | |
| Ingredients | tablet | g/batch | tablet | g/batch | tablet | g/batch |
| TFV RD | 63.69 | 21.23 | 62.89 | 838.6 | 61.35 | 818.0 |
| Microgranules | | | | | | |
| Rapidly | 61.56 | 20.52 | 62.36 | 831.4 | 63.90 | 852.0 |
| Dispersing | | | | | | |
| Granules | | | | | | |
| Crospovidone | 7.5 | 2.5 | 7.5 | 100.0 | 7.5 | 100.0 |
| Avicel PH101 | 15.0 | 5.0 | 15.0 | 200.0 | 15.0 | 200.0 |
| Sodium stearyl | 2.3 | 0.75 | 2.3 | 30.0 | 2.3 | 30.0 |
| fumarate | | | | | | |
| Total | 150.0 | 50.0 | 150.0 | 2000 | 150.0 | 2000 |
| Parameter | | | | | | |
| Compression | Formula Ex. 2BD | | | Formula. Ex. 2CD | | |
| Force (4 kN) | Start | Mid | End | Start | Mid | End |
| Weight (mg) | 151 | 150 | 149 | 149 | 151 | 150 |
| Thickness, mm | 3.53 | 3.54 | 3.54 | 3.52 | 3.53 | 3.52 |
| Hardness, N | 39 | 34 | 29 | 38 | 32 | 28 |
| Friability | 0.05% | 0.11% | 0.13% | 0.16% | 0.15% | 0.20% |

*Tablets compressed using a Carver press.

E: Single and Multi-Dose PK Study of Tenofovir RDT Vaginally Administered in Rabbits:

The objective of this study was to evaluate the pharmacokinetics of tenofovir after a single dose or seven daily doses when administered in rapid dissolve tablet form in female rabbits with a minimum body weight of 2.5 kg (n=6 in each group; blood sampled at 0, 0.5, 1, 2, 4, 8, and 24 hrs post dosing day 1 or 7 in group 1 & 3 and at 0, 0.5, 1.0, 1.5, and 2 hrs post dosing day 1 or 7 in group 2 & 4). Prior to test article administration, acepromazine maleate (0.3-0.5 mg/kg or to effect) was administered via subcutaneous administration to mildly sedate the animal. Once sedated, a single tablet was inserted into the abdominal vagina of each animal (approximately 8 cm) with an 18 catheter. No lubrication was used as this may affect tablet absorption. A detailed clinical examination of each animal was performed daily during the study. Observations included, but was not limited to, evaluation of the skin, fur, eyes, ears, nose, oral cavity, thorax, abdomen, external genitalia, limbs and feet, respiratory and circulatory effects, autonomic effects such as salivation, nervous system effects including tremors, convulsions, reactivity to handling, and atypical behavior. Complete necropsy examinations were performed for groups 1 and 3 on day 1 or 7 at 2 hrs or groups 2 and 4 at 24 hrs post dosing, under yellow lighting using procedures approved by a veterinary pathologist on all animals found dead, euthanized extremis, or euthanized at each scheduled necropsy in accordance with current SOP. Iliac lymph nodes were collected under yellow lighting for determination of total tenofovir concentrations. Samples of Weck-Cel® sponge were prepared by placing each Weck-Cel® sponge into the vagina in order to absorb secretions from the vagina for determination of total tenofovir concentrations.

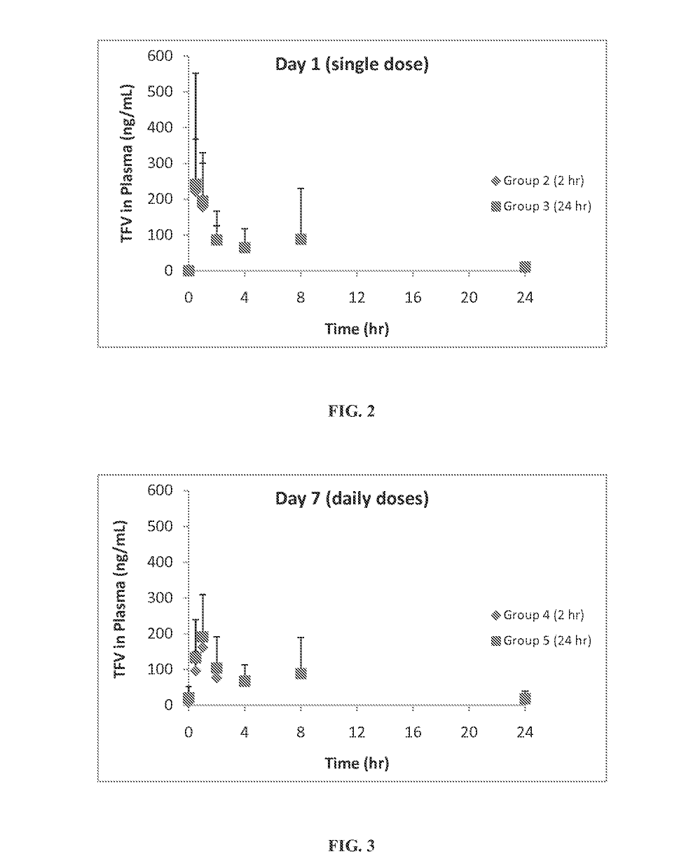

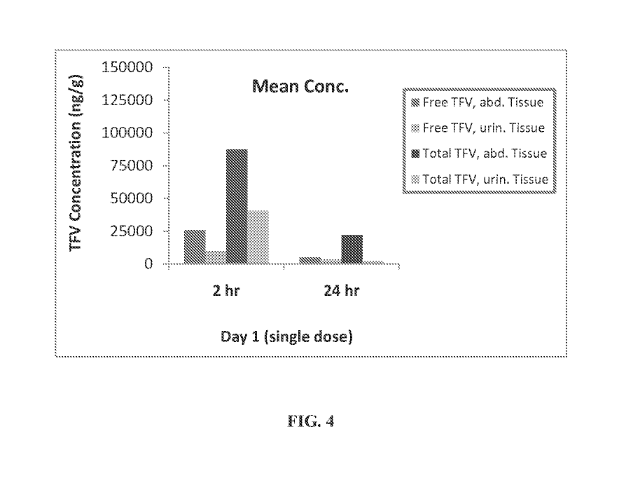

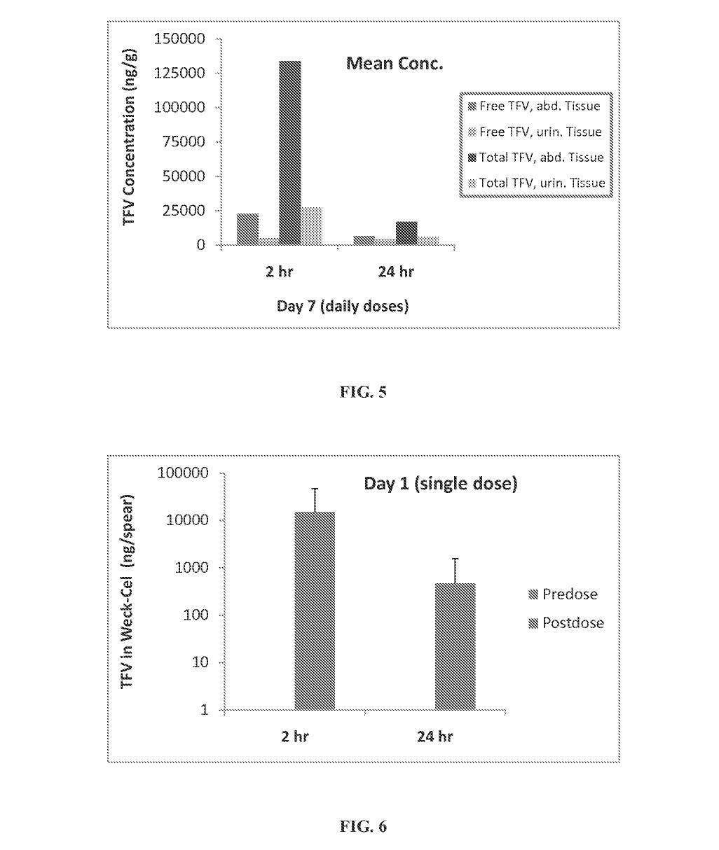

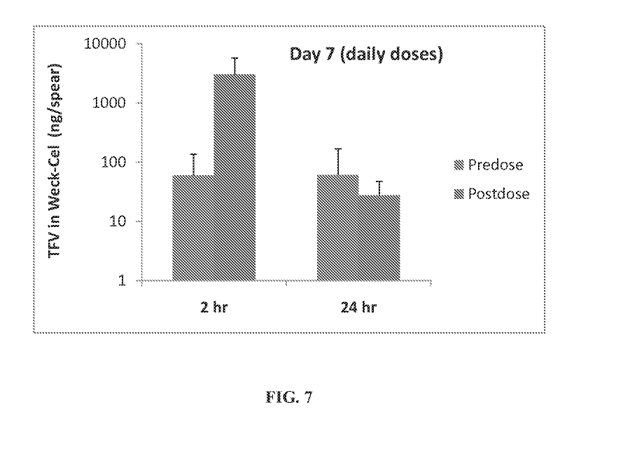

FIG. 2 shows the mean tenofovir plasma concentration-time profiles following insertion of a single tenofovir RDT (i.e., RDTs of Ex. 2CD) into the vaginal cavity of female rabbits while FIG. 3 shows the corresponding tenofovir concentrations following multi-dose (7 once-daily dosing) administration. FIG. 4 shows the mean free and total tenofovir contents of the abdominal and vaginal tissues at 2 and 24 hrs post dosing following insertion of a single tenofovir RDT into the vaginal cavity of female rabbits while FIG. 5 shows the corresponding fenofovir concentrations following multi-dose (7 once-daily dosing) administration, FIG. 6 shows the mean predose and postdose tenofovir concentrations in Weck-Cel® at 2 and 24 hrs post dosing following insertion of a single tenofovir RDT into the vaginal cavity of female rabbits while FIG. 7 shows the corresponding predose and postdose tenofovir concentrations in Weck-Cel® following multi-dose (7 once-daily dosing) administration.

Further preclinical comparative PK studies in rabbits dosing 40 mg as rapid dissolve tablets (four 10 mg tablets) versus 40 mg as 1% tenofovir gels (4 ml) suggest that the rapid dissolve tablet form provides equivalent tissue concentrations as compared to the 1% TFV gel. Unlike the gel dosage form which requires the use of a vaginal applicator and accompanying packaging, the rapid dissolve tablet technology makes it very attractive in terms of increased portability and potential reductions in manufacturing costs. Additionally, the technology affords privacy and convenience of medication administration.

### Example 3

A: RD Microgranules Comprising Tenofivir:

The high shear granulator GMX-25 is charged with tenofovir (TFV; 829.2 g), mannitol (Pearlitol 25 with a mean particle size of less than 30 μm; 7829.2 g), hydroxyethylcellulose (NITROSOL-HEC 250L; 91.6 g), and crospovidone (200 g). The contents of the product bowl are well mixed with the impeller speed set at 150 RPM for 2 minutes. The powder mixture is granulated by spraying purified water at a spray rate of about 100 g/min at the following processing parameters; spray nozzle pore size—0.085″; impeller setting: speed—325 RPM, time—5.5 min; Chopper setting speed—High, time—5.5 min. After 5 minute, stop spraying, allow the granulation to continue to mix for another 30 sec before stopping the granulator and scrape the bowl, chopper blade and impeller blades. The spraying is continued to spray about 630 g of water and the contents of the product bowl are mixed for another 2 minutes before discharging the contents of the product bowl. The product bowl of pre-heated Glatt GPCG 5 is charged with the moist granulation which is dried for a loss on drying of less than 2% as determined using a Computrac Moisture Analyzer at 85° C. The drying conditions in the Glatt are as follows: product support screen—200 mesh; inlet air temperature—42° C.; inlet air volume—40 cfm; Desiccant Wheel—ON.

B: RD Microgranules Comprising Emtricitabine:

Emtricitabine (FTC, 682.9 g) is slowly added to a mixture of ethanol (3974 g) and purified water (995 g) in a stainless steel container while continuously stirring to dissolve. Next, low substituted hydroxyethylcellulose (Natrosol HEC-250L; 80.1 g) is added to disperse/dissolve while stirring. Preheated Glatt GPCG 3 is charged with deagglomerated mannitol (2737 g) and fluidized. The RD microgranules are prepared by spraying the solution at the following conditions: product support screen—200 mesh; nozzle tip size—1.2 mm; atomization pressure—2 bar; inlet air temperature—72° C.; product temperature—38±1° C.; fluidization air flow—40 cfm; spray rate—40 mL/min; desiccant wheel—ON. Upon completion of spraying, the FTC RD microgranules are dried at the inlet air temperature set at 42° C. for a loss on drying to about 1% by weight.

C. RDTs (40-mg TFV/40-mg FCT)

RDT tablet formulations containing TFV RD microgranules of Ex. 3A and FCT RD microgranules of Ex. 3B alone, or their mixtures thereof are first blended with other pharmaceutical excipients including the lubricant, sodium stearyl fumarate and are compressed (see Table 3 for compositions) on a Beta tablet press equipped with partial tooling at a compression force of 4-6 kN and turret speed of 15 RPM. Samples are collected at the start, mid, and end of each run for testing for in-process tablet properties.

The Manesty Beta press equipped with eight (8) 12 mm round, lozenge tooling having no embossing is set up to the following parameters for 40 mg TFV/40 mg FCT RDTs:

| | | |
|---|---|---|
| Tablet weight: total(10)—5.00 g; | Nominal: 500 mg; | Range: 460-540 mg |

| | |
|---|---|
| Fill weight setting: 8 mm | Pre-compression setting: 6 mm |
| (or 6 mm for TFV); | |
| Main compression: 3.3 mm | Force feeder setting: 3. |
| (or 2.85 mm for FTV) | |

The press was set to run at 25 rpm and after a few die table/turret rotations, 10 tablets are collected before stopping the press. Ten tablets are collected for determining the weight of 10 tablets and are inspected for tablet's appearance (picking, capping, etc). The tablet press is adjusted, as necessary, in order to produce tablets that meet the specifications listed above for weight, thickness and hardness, and results are recorded on the production batch record. If required, the parameters are readjusted as necessary to produce tablets that meet the friability specifications listed above

At the beginning, middle and end of the process run, thirty five (35) tablets are sampled for in-process testing. Ten tablets are tested for weight, hardness and thickness, and 6.5 g tablets are tested for friability. Results are recorded on the production batch records. The rest of the samples are combined in a properly labeled container as part of the composite sample. Each in process test point will utilize a separate container for analytical testing.

Using similar settings, rapidly disintegrating tablets of 40-mg TFV/20-mg FCT, 20-mg TFV/40-mg FCT, 40-mg TFV or 40-mg FCT are compressed from their respective compression mixes.

TABLE 3
Compositions of RDTs (40 mg TFV; 40 mg FCT; 40 mg TFV/40 mg FCT;
40 mg TFV/20 mg FCT; 20 mg TFV/40 mg FCT)

| | Formula | | | | | |
|---|---|---|---|---|---|---|
| | Ex. 3C | | Formula | | | |
| | 40 mg TFC/ | | Ex. 3C | | Formula Ex. 3C | |
| | 40 mg FCT | | 40 mg/ | 40 mg/ | 40 mg | 40 mg |
| | mg/ | | 20 mg | 20 mg | FTV | FCT |
| Ingredients | tablet | g/batch | g/batch | g/batch | mg/tablet | g/batch |
| TFV RD | 205.0 | 1148.0 | 1148.0 | 574.0 | 1148.0 | — |
| Microgranules | | | | | | |
| FCT RD | 205.0 | 1148.0 | 574.0 | 1148.0 | — | 1148.0 |
| Microgranules | | | | | | |
| Rapidly | 17.5 | 98.0 | 672.0 | 672.0 | 1288.0 | 1176.0 |
| Dispersing | | | | | | |
| Granules | | | | | | |
| Crospovidone | 17.5 | 98.0 | 980.0 | 98.0 | 56.0 | 140.0 |
| Avicel PH101 | 50.0 | 280.0 | 280.0 | 280.0 | 280.0 | 280.0 |
| Sodium stearyl | 5.0 | 28.0 | 28.0 | 28.0 | 28.0 | 56.0 |
| fumarate | | | | | | |
| Total | 500.0 | 2800 | 2800.0 | 2800 | 2800.0 | 2800 |

### Example 4

A: RD Microgranules Comprising Metronidazole:

Vinylpyrrolidone-vinyl acetate copolymer (e.g., Kollidon® VA 64 from BASF; 50 g) is slowly added to a mixture of ethanol and purified water in a stainless steel container while continuously stirring to dissolve. Next, metronidazole (180 g) is added to dissolve while stirring. Preheated Glatt GPCG 3 is charged with deagglomerated mannitol (770 g) and crospovidone (50 g) and fluidized. The RD microgranules are prepared by spraying the solution at the following conditions: product temperature—34±1° C.; fluidization air flow—4 CFM; spray rate—8-12 mL/min. Upon completion of spraying, the RD microgranules are dried for a loss on drying to about 1% by weight.

B: RD Microgranules Comprising Clotrimazole:

Hydroxyethylcellulose (50 g) is slowly added to a mixture of ethanol and purified water in a stainless steel container while continuously stirring to dissolve. Next, metronidazole (180 g) is added to dissolve while stirring. Preheated Glatt GPCG 3 is charged with deagglomerated mannitol (720 g) and crospovidone (50 g) and fluidized. The RD microgranules are prepared by spraying the solution at the following conditions: product temperature—34±1° C.; fluidization air flow—4 CFM; spray rate—8-12 mL/min. Upon completion of spraying, the RD microgranules are dried for a loss on drying to about 1% by weight.

C: RD Microgranules Comprising Propranolol HCl:

Vinylpyrrolidone-polyvinyl acetate copolymer (e.g., Kollidon® VA 64 from BASF; 50 g) is slowly added to purified water in a stainless steel container while continuously stirring to dissolve. Next, propranolol HCl (240 g), a non-specific beta-blocker exhibiting extensive hepatic metabolism, is added to dissolve while stirring. Preheated Glatt GPCG 3 is charged with deagglomerated mannitol (560 g) and low-substituted hydroxyethylcellulose (50 g) and fluidized. The RD microgranules are prepared by spraying the solution at the following conditions: product temperature—38±2° C.; fluidization air flow—10 CFM; spray rate—10-20 mL/min. Upon completion of spraying, the RD microgranules are dried for a loss on drying to about 1% by weight.

D. RDTs Containing Metronidazole, Clotrimazole, or Propranolol HCl

RDT tablets (Formula Ex. 4AD: Metronidazole RDTs, Formula Ex. 4BD: Clotrimazole RDTs, and Formula Ex. 4CD: Propranolol HCl RDTs) containing required amounts of Metronidazole RD microgranules of Ex. 4A, Clotrimazole RD microgranules of Ex. 4B, or Propranolol HCl RD microgranules of Ex. 4C, rapidly dispersing microgranules from Ex. 1D above at 5-15% by weight, microcrystallinecellulose (Avicel PH101 at 5-10% by weight), low-substituted hydroxypropylcellulose at 2-5% by weight, sodium stearyl fumarate, at 1% by weight are compressed on a Hata tablet press equipped with appropriate tooling at different compression forces and at different turret speeds. Samples are collected at the start, mid, and end of the run for testing for in-process tablet properties to establish the robustness of the manufacturing processes of each tablet formulation.

The skilled artisan will recognize that the above procedures and compositions can be suitably modified to provide the appropriate dose of drug(s) whose rapid dissolve tablet formulations for vaginal administration are required.

While the invention has been described in connection with the specific embodiments herein, it will be understood that it is capable of further modifications and this application is intended to cover any variations, uses, or adaptations of the invention following, in general, the principles of the invention and including such departures from the present disclosure as come within known or customary practice within the art to that the invention pertains and as may be applied to the essential features hereinbefore set forth and as follows in the scope of the appended claims.

All documents, patents, patent applications, and publications cited herein are incorporated by reference in their entirety for all purposes.

1. A pharmaceutical composition in the form of rapid dissolve microgranules for vaginal administration comprising:
(a) a therapeutically effective amount of one or more vaginally active drugs;
(b) a polymeric excipient having a dual property of acting as a binder as well as a bioadhesive material;
(c) a sugar alcohol, a saccharide, or a mixture thereof; and
(d) a disintegrant;
wherein said pharmaceutical composition rapidly disintegrates upon insertion into the vaginal cavity of a patient or subject forming a viscous drug-containing suspension that rapidly, widely spreads coating the vaginal mucosa.

2. The pharmaceutical composition of claim 1, wherein said therapeutically effective drug is selected from the group consisting of antifungal agents, antibacterial agents, antimicrobial agents, antiviral agents, anti-infectives, spermicides, steroids, hormones, analgesics including non-steroidal antiflammatory drugs, cardiovascular agents, calcium channel blockers, beta blockers, antiarrhythmics, antihypertensives, diuretics, general, coronary, peripheral and cerebral vasodilators, antimigraine agents, erectile dysfunction agents, central nervous system stimulants, sedatives, hypnotics, immunosuppressives, muscle relaxants, orally active drugs exhibiting significant first-pass effects, β-adrenergic agonists, tranquilizers, antioxidants, vitamins, antitrichomonial agents, antiprotozoan agents, antimicoplasm agents, antiretroviral agents, nucleoside analogues, reverse transcriptase inhibitors, protease inhibitors, contraceptive agents, sulfa drugs, sulfonamides, sulfones, peptides, proteins, growth hormones, and luteinizing-hormone-releasing hormone, or mixtures or combinations thereof.

3. The pharmaceutical composition of claim 2, wherein said therapeutically effective drug is a reverse transcriptase inhibitor, nucleoside or nucleotide reverse transcriptase inhibitor, non-nucleotide reverse transcriptase inhibitor, protease inhibitor selected from the group consisting of apricitabine, entecavir, emtricitabine, tenofovir, abacavir, adefovir, nevirapine, delavirdine, efavirenz, UC-781, MKC-442, quinoxaline HBY 097, DMP 266, indinavir, amprenavir, darunavir, lotinavir, nelfinavir, ritonavir, sequinavir, atazanavir, tipranavir, elvitegravir, and MK-2048, or their pharmaceutically acceptable salts, prodrugs, or mixtures or combinations thereof.

4. The pharmaceutical composition of claim 2, wherein said therapeutically effective drug is an antifungal agent selected from the group consisting of butoconazolenystatin, oxiconazle, fluconazole, posaconazole, clotrimazole, and ketoconazole, or their pharmaceutically acceptable salts, or mixtures or combinations thereof.

5. The pharmaceutical composition of claim 2, wherein said therapeutically effective drug is an antibacterial agent selected from the group consisting of clindamycin, sulfonamides, erythromycin, clarithromycin, azithromycin, doxycycline, metronidazole, macrolide antibiotics, quinolones, cephalosporins, cefoxitin, and ceftriaxone, or mixtures or combinations thereof.

6. The pharmaceutical composition of claim 2, wherein said therapeutically effective drug is an antiviral agent selected from the group consisting of penciclovir, acyclovir, genciclovir, and valaciclovir, or mixtures or combinations thereof.

7. The pharmaceutical composition of claim 2, wherein said therapeutically effective drug is a cardiovascular agent selected from the group consisting of verapamil, propranolol, metoprolol, diltiazem, isradipine, felodipine, nifedipine, and nicardipine, or mixtures or combinations thereof.

8. The pharmaceutical composition of claim 2, wherein said therapeutically effective drug is a non-steroidal anti-inflammatory drug selected from the group consisting of aspirin, ibuprofen, indomethacin, sulindac, naproxen, and nebumetone, or mixtures or combinations thereof.

9. The pharmaceutical composition of claim 1, further comprising at least one surfactant selected from the group consisting of DL-alpha tocopherol, CAPTEX 200, Tween 20 Tween 80, Vitamin E TPGS, Capryol 90, CREMOPHOR EL, CARBITOL, PEG 400, lecithin, BRIJ 92, LABRASOL, triacetin, sodium lauryl sulfate, ethylene glycol monostearate, polysorbates PLURONIC®, GELUCIRE®, LABRAFIL®, LABRASOL®, IMWITOR®, sodium lauryl salicylate, and sodium dodecyl sulfate, or mixtures thereof.

10. The pharmaceutical composition of claim 1, further comprising at least one lipid selected from the group consisting of lecithins, hydrogenated lecithins, lysolecithin, hydrogenated lysolecithins, lysophospholipids and derivatives thereof, phospholipids and derivatives thereof, salts of alkylsulfates, salts of fatty acids, sodium docusate, stearyl alcohol, glyceryl palmitostearate, mixtures of mono-, di-, and tri-esters of glycerol, mono- and di-esters of PEG, and free PEG, or mixtures thereof.

11. The pharmaceutical composition of claim 1, further comprising a population of rapidly-dispersing microgranules each having an average particle size of not more than about 400 μm and comprising (1) a disintegrant and (2) a sugar alcohol or a saccharide, wherein said sugar alcohol or saccharide each has an average particle size of not more than about 30 μm.

12. The pharmaceutical composition of claim 11, wherein the ratio of rapid dissolve drug-containing microgranules to rapidly-dispersing microgranules ranges from about 50:1 to about 1:2.

13. The pharmaceutical composition of claim 11, wherein the rapidly-dispersing microgranules comprise a disintegrant selected from the group consisting of crosslinked polyvinylpyrrolidone, sodium starch glycolate, crosslinked carboxymethylcellulose of sodium, and low-substituted hydroxypropylcellulose, or mixtures thereof.

14. The pharmaceutical composition of claim 11, wherein the rapidly-dispersing microgranules comprise a sugar alcohol selected from the group consisting of arabitol, erythritol, glycerol, isomalt, lactitol, maltitol, mannitol, sorbitol, and xylitol, or combinations thereof.

15. The pharmaceutical composition of claim 1, further defined as a rapid dissolve tablet that disintegrates in about 60 seconds when tested by USP method <701> disintegration time.

16. The pharmaceutical composition of claim 15, further defined as a rapid dissolve tablet comprising said rapidly-dispersing microgranules with an average particle size of not more than about 400 μm, further comprising a disintegrant and a sugar alcohol or a saccharide or a combination thereof, each having an average particle size of not more than about 30 μm, wherein said rapid dissolve tablet exhibits the following properties:
i) a friability of not more than 1% by weight; and
ii) sufficient tablet hardness suitable for packaging in blisters or bottles for storage, transportation, commercial distribution, and end use.

17. A method for the preparation of the pharmaceutical composition of claim 1, comprising:
a) preparing rapid dissolve microgranules comprising:
i. at least one therapeutically effective drug selected from the group consisting of antifungal agents, antibacterial agents, antimicrobial agents, antiviral agents, anti-infectives, spermicides, steroids, hormones, analgesics including non-steroidal antiflammatory drugs, cardiovascular agents, calcium channel blockers, beta blockers, antiarrhythmics, antihypertensives, diuretics, general, coronary, peripheral and cerebral vasodilators, antimigraine agents, erectile dysfunction agents, central nervous system stimulants, sedatives, hypnotics, immunosuppressants, muscle relaxants, orally active drugs exhibiting significant first-pass effects, β-adrenergic agonists, tranquilizers, antioxidants, vitamins, antitrichomonial agents, antiprotozoan agents, antimicoplasm agents, antiretroviral agents, nucleoside analogues, reverse transcriptase inhibitors, protease inhibitors, contraceptive agents, sulfa drugs, sulfonamides, sulfones, peptides, proteins, growth hormones, and luteinizing-hormone-releasing hormone, or mixtures or combinations thereof;
ii. at least one sugar alcohol, saccharide, or a mixture thereof, selected from the group consisting of mannitol; and
iii. polymeric excipient selected from the group consisting of low-substituted hydroxyethylcellulose, hydroxypropylcellulose, hypromellose, polycarboxylic acids, polyvinylpyrrolidone, vinylpyrrolidone-polyvinyl acetate copolymer, ethylene glycol 6000-vinylcaprolactam-vinyl acetate copolymer, polyvinyl alcohol, polyethylene oxide, poly(lactic co-glycolic acid), polyamide, alginic acid salts, carrageenan, chitosan, and cellulosic gum.

18. The method of claim 17, further comprising:
b) preparing rapidly dispersing microgranules comprising:
i. at least one sugar alcohol, saccharide, or a mixture thereof, selected from the group consisting of arabitol, erythritol, glycerol, isomalt, lactitol, maltitol, mannitol, sorbitol, and xylitol, or combinations thereof; and
ii. at least one disintegrant selected from the group consisting of crosslinked polyvinylpyrrolidone, sodium starch glycolate, crosslinked carboxymethylcellulose of sodium, and low-substituted hydroxypropylcellulose, or mixtures thereof, at a ratio of 90:10 to 99:1;
c) blending rapid dissolve microgranules from step a), rapidly dispersing microgranules from step b), and at least one pharmaceutically acceptable excipient selected from the group consisting of fillers, disintegrants, and lubricants selected from the group consisting of microcrystalline cellulose, crospovidone, low-substituted hydroxypropylcellulose, magnesium stearate, and sodium stearyl fumarate; and
d) compressing the product of c) into rapid dissolve tablets via compressing on a rotary tablet press;
wherein the rapid dissolve tablet rapidly disintegrates upon insertion into the vagina of a patient or subject in need thereof, forming a viscous drug-containing suspension that rapidly, widely spreads, coating the vaginal mucosa.

19. The method of claim 17, further comprising:
a) preparing rapid dissolve microgranules comprising tenofovir, mannitol, low-substituted hydroxyethylcellulose, and optionally crospovidone and bioadhesive chitosan;
b) granulating mannitol and crospovidone each having an average particle size of not more than about 30 μm to produce rapidly dispersing microgranules;
c) blending the rapid dissolve microgranules from step a), rapidly dispersing microgranules from step b), and microcrystalline cellulose, crospovidone, and sodium stearyl fumarate to form a blend; and
d) compressing the blend of step c) into rapid dissolve tablets using a rotary tablet press;
wherein each rapid dissolve tablet disintegrates in about 60 seconds when tested by USP method <701> disintegration time.

20. The method of claim 18 wherein step d) further comprises compressing the blend of step c) into rapid dissolve tablets using a rotary tablet press equipped with an external lubrication system to lubricate the dies and punches prior to compression with magnesium stearate.

21. The method of claim 19, wherein step a) further comprises preparing rapid dissolve microgranules comprising emtricitabine, mannitol, low-substituted hydroxyethylcellulose, and optionally crospovidone and bioadhesive carbopol to prepare rapid dissolve tablets containing therapeutically effective amounts of both tenofovir and emtricitabine for administration into the vagina of a patient or subject in need thereof.

22. A method comprising administering a pharmaceutical composition of claim 1 containing a therapeutically effective amount of one or more vaginally active drugs into the vagina of a patient or subject in need thereof.

23. The method of claim 22, wherein the vaginally active drugs comprise therapeutically effective amounts of tenofovir and emtricitabine for the treatment of HIV infection.
